(19) United States
(12) Patent Application Publication
(10) Pub. No.: US 2008/0245764 A1
(43) Pub. Date: Oct. 9, 2008

(54) Method for producing a device including an array of microneedles on a support, and device producible according to this method

(76) Inventors: Tjalf Pirk, Leonberg (DE); Michael Stumber, Korntal-Muenchingen (DE); Joachim Rudhard, Leinfelden-Echterdingen (DE); Ando Feyh, Tamm (DE); Christina Leinenbach, Ensdorf (DE); Christian Mauerer, Leonberg (DE)

Correspondence Address: KENYON & KENYON LLP, ONE BROADWAY, NEW YORK, NY, 10004, US

(21) Appl. No.: 12/009,498

(22) Filed: Jan. 18, 2008

(30) Foreign Application Priority Data
Jan. 19, 2007 (DE) 10 2007 002 832.8

(51) Int. Cl. C23F 1/00; A61M 5/158

(52) U.S. Cl. 216/2; 216/11; 604/272

(57) ABSTRACT

A method for producing a device which is suitable for delivering a substance into or through the skin and includes an array of microneedles developed out of an Si semiconductor substrate, the microneedles being affixed on and/or inside a flexible support made from a polymer material. A device producible by this method.

### FIELD OF THE INVENTION

The present invention relates to a method for producing a device which is suitable for delivering a substance into or through the skin, the device including an array of microneedles developed out of a Si semiconductor substrate and affixed on and/or inside a flexible support made from a polymer material. Furthermore, the present invention relates to a device which is suitable for delivering a substance into or through the skin, the device being producible according to the method of the present invention.

### BACKGROUND INFORMATION

The delivery of substances into the body through the skin is usually carried out invasively in the form of an injection using needles and syringes. These methods can be painful for the patient, require skilled medical personnel, and may lead to injuries, bleeding or infections. To spare patients an injection, attempts have been made for some time to deliver pharmaceutical and other substances by transdermal application. One possibility consists of the use of microneedles, with whose aid a permeability of the stratum corneum of the skin is able to be achieved, for instance by producing micro-injuries by slight tearing or perforation. This method has the advantage of being essentially painfree and of not causing any bleeding, and it allows the active ingredients to gain access to the tissue layers lying underneath.

Often, a multitude of microneedles is used such as in the form of an array. An array denotes a system of microneedles on a support. The microneedles are removable once the skin has been torn slightly or perforated, and an active ingredient depot such as a plaster may be applied onto the skin, whereupon the active ingredients released therefrom are able to pass through the skin more easily. As an alternative, the active ingredient may be applied directly via the needles.

Microneedles are often produced on the basis of a silicon semiconductor. They can then be detached or, if arrays of microneedles are to be used, a suitable number of microneedles may remain on a portion of the semiconductor as support and be used in the form of an array.

These arrays are generally made from a single material, such as silicon, polymer or metal. Arrays made of silicon are inflexible, however, and not suitable for adapting themselves to irregularities or uneven or rounded structures of the skin.

No further methods for producing devices that include microneedles and supports made from different materials are known.

### SUMMARY OF THE INVENTION

In contrast, the method of the present invention for producing a device which is suitable for delivering a substance into or through the skin and includes an array of microneedles developed out of an Si semiconductor substrate and affixed on and/or inside a flexible support made from a polymer material, has the advantage of providing a method that requires a limited number of processing steps.

According to the exemplary embodiments and/or exemplary methods of the present invention, this is achieved in that the method for producing a device, which includes a system of microneedles developed out of a Si semiconductor substrate and affixed on and/or inside a flexible support made from a polymer material, encompasses the following steps:
a) Supplying a Si semiconductor substrate (1);
b) Depositing and patterning a masking layer on the surface of the Si semiconductor substrate (1), the masking layer having openings through which the etching reagent acts on the Si semiconductor substrate (1);
c) Developing microneedles (2) out of the Si semiconductor substrate (1) with the aid of micromechanical patterning techniques through the masking layer, the microneedles tapering from the outer surface in the direction of the base of the Si semiconductor substrate (1), using wet or dry chemical etching methods;
d) Optionally, removing the masking layer;
e) Optionally, rendering the Si semiconductor substrate (1) and/or the microneedles (2) porous;
(f) Depositing a coherent polymer support layer (3) on the microneedle surfaces facing away from the Si semiconductor substrate (1);
(g) Detaching the microneedles (2) from the Si semiconductor substrate (1).

In an advantageous manner, the method according to the present invention for producing a device including an array of microneedles, which are developed out of an Si semiconductor substrate and affixed on and/or inside a flexible support made from a polymer material, has a limited number of masking steps.

Furthermore, the method according to the present invention permits a simple and cost-effective production of flexible devices that adapt themselves to the shape of the patient's patch of skin onto which they are applied and make it possible to provide a uniform penetration depth of the microneedles.

Exemplary embodiments of the present invention are illustrated in the drawing and elucidated in greater details on the basis of FIGS. 1 through 8.

### BRIEF DESCRIPTION OF THE DRAWINGS

FIG. 1 shows a view of the microneedles on a Si semiconductor substrate.

FIG. 2 shows a view of the microneedles; the microneedles have been partially embedded in the support via the region that has a wider cross-section and have been detached from the Si semiconductor substrate.

FIG. 3 shows a view of the microneedles on a Si semiconductor substrate, which has been rendered porous in the region of the microneedles and a layer lying underneath.

FIG. 4 shows a view of the microneedles on a Si semiconductor substrate; the microneedles are partially embedded in the support via the region that has a wider cross-section, and accesses to the microneedles may be provided in the support, as indicated by way of example for a few microneedles.

FIG. 5 shows a view of the microneedles on a Si semiconductor substrate; the microneedles are partially embedded in the support via the region that has a wider cross-section, and a sacrificial resist is provided between Si semiconductor substrate and support.

FIG. 6 shows a view of the microneedles on a Si semiconductor substrate; the microneedles are partially embedded in the support via the region that has a wider cross-section, and a spacing foil is provided between Si semiconductor substrate and support.

FIG. 7 shows a view of the microneedles on a Si semiconductor substrate; the microneedles are partially embedded in the support via the region that has a wider cross-section, and a spacer developed out of the Si semiconductor substrate is provided between Si semiconductor substrate and support.

FIG. 8 shows a view of the microneedles partially embedded in the support via the region that has a wider cross-section, and of the Si semiconductor substrate after being detached.

### DETAILED DESCRIPTION

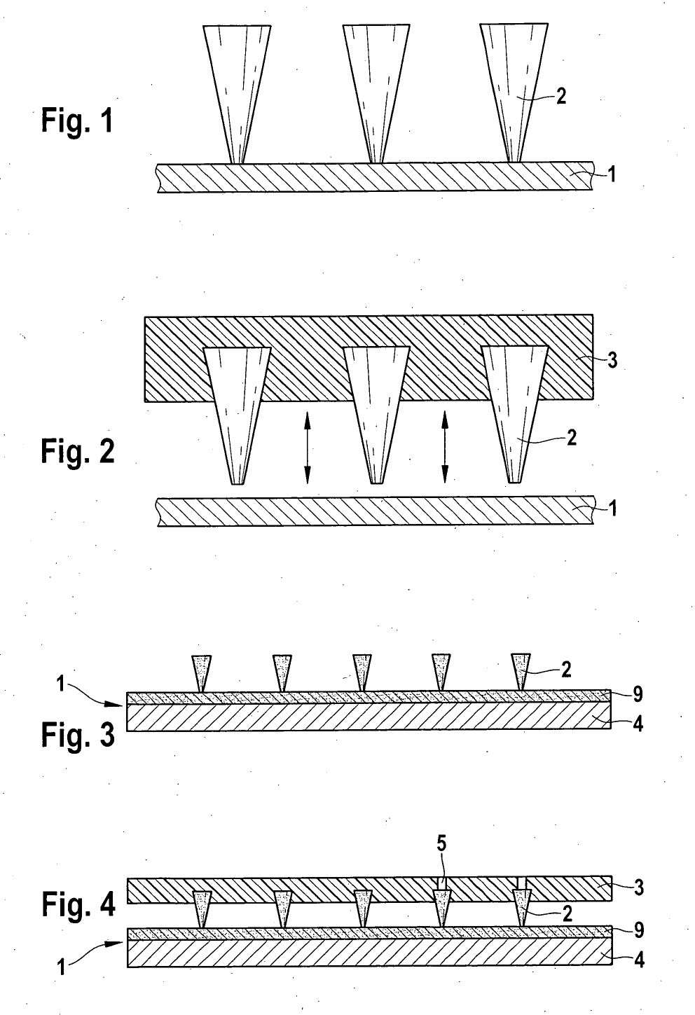

FIG. 1 shows tapering microneedles 2 having a negative profile on an Si semiconductor substrate 1. As illustrated according to FIG. 2, microneedles 2 were embedded in a polymer support 3 and detached from the Si semiconductor substrate 1.

Microneedles 2 having a negative profile as illustrated in FIG. 3 were produced according to the method described in the patent DE 42 41 045; this method, which is known as Bosch process, was modified according to the exemplary embodiments and/or exemplary methods of the present invention by reducing the effectiveness of the passivation steps. One layer of a silicon wafer was first rendered porous. With the aid of PECVD (plasma-enhanced chemical vapor deposition) methods, a silicon-oxide layer was then produced on porosified Si semiconductor substrate 1, and a photoresist AZ® 5433 (Clariant) thereupon deposited on this surface. The masking layer was patterned photolithografically, and openings were provided for the passage of the etching reagent. Etching was implemented using SF₆ as etching reagent and C₄F₈ as passivator in alternation, the ratio of etching time and passivation time being 3:1. Since the side walls were attacked more heavily in the lower region during the patterning steps, microneedles 2 having a negative profile were produced. FIG. 3 shows porosified microneedles 2 on a likewise porosified layer 9 of Si semiconductor substrate 1, while a layer 4 not rendered porous is made of the originally monocrystalline silicon.

Using hot-stamping, microneedles 2 then were partially embedded in a polymer support 3 via their region having a wider cross-section, as illustrated in FIG. 4. Accesses 5 to microneedles 2 may be provided in support 3, as sketched for some of the microneedles by way of example.

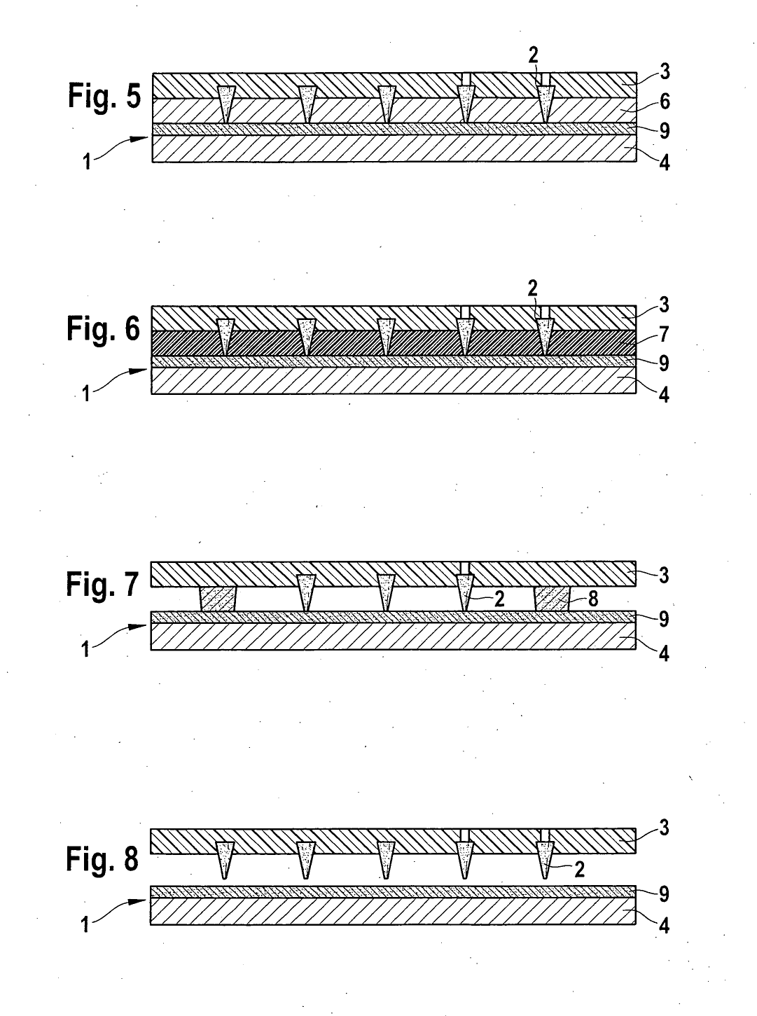

To adjust the length of the projecting section of the microneedles, a sacrificial resist layer 6 was provided as spacer between Si semiconductor substrate 1 and support 3 according to FIG. 5, the layer thickness corresponding to the length of the microneedles subsequently projecting from the support. In alternative specific embodiments as illustrated according to FIG. 6, a polymer spacing foil 7 may be provided. In further alternative specific embodiments as illustrated according to FIG. 7, a non-etched region of the Si semiconductor substrate may be used as spacer in the form of a frame 8.

After microneedles 2 were compression-molded to support 3, the tips of the microneedles were detached from Si semiconductor substrate 1. FIG. 8 shows the device of microneedles 2 partially embedded in support 3 via the region having a wider cross-section, after being detached.

Further embodiments of the device according to the present invention are described and elucidated in greater detail in the following text.

Silicon wafers may be employed as especially suitable Si semiconductor substrate. It is possible, for instance, to use commercially obtainable silicon wafers. Utilizable are silicon wafers having a passivation of the silicon surface, which may be by an oxide layer, for instance with the aid of vapor-phase deposition by PECVD (plasma-enhanced chemical vapor deposition) methods.

A masking layer is produced on the surface of the Si semiconductor substrate. A photo-resist layer having positive or negative exposure characteristics may be used, which is subsequently patterned, which may be with the aid of lithographic methods, especially photo-lithographic methods.

Suitable are, for example, liquid resists such as a photo resist. For instance, photo resists known as AZ® 5433 and obtainable from the Clariant GmbH company are able to be utilized.

It may be provided that a silicon-oxide layer be applied as hard-surface mask prior to applying the photo-resist layer, which then is patterned using photo-lithographic methods. SiO₂ or Si₃N₄ layers are also suitable as masking layer. The masking layer may also be made from other substances such as SiC. Likewise usable as masking layer within the scope of the method according to the present invention are layers that are able to be deposited by CVD (chemical vapor deposition), e.g., silicon-oxide layers or other suitable resist layers.

The masking layer includes openings through which the etching reagent acts on the Si semiconductor substrate. In specific embodiments the openings in the masking layer are separated by webs developed in the masking layer in the form of a grid. The webs may have a distance relative to each other in the range from ≧100 μm to ≦250 μm, which may be in the range from ≧150 μm to ≦200 μm. The webs may have a width ranging from ≧5 μm to ≦40 μm, which may range from ≧10 μm to ≦30 μm.

In exemplary embodiments the points of intersection of the webs are widened so as to create areas having a diameter in the range from ≧30 μm to ≦60 μm, which may be in the range from ≧40 μm to ≦50 μm. The areas may have a round form, but they may also have an angular form, e.g., a square, rectangular, or octagonal form, in which case the diameter means the average diameter.

The webs formed in the masking layer are able to be undercut by etching. This may offer the advantage of providing the microneedles in the form of individual microneedle structures. It may also be provided that the webs are undercut only slightly by etching and the microneedles are provided in the form of a coherent array.

In exemplary embodiments, tapered microneedles are developed out of the Si semiconductor substrate through the masking layer by micromechanical patterning techniques with the aid of wet or dry chemical etching methods. The micromechanical patterning techniques may be dry chemical etching methods or deep-etching methods, and may especially be deep-etching methods using a plasma.

The methods may be DRIE (deep reactive ion etching) methods modified according to the present invention, or etching methods using alternating etching and passivation steps. The method modified according to the present invention may comprise two complementary etching steps, i.e., etching using SF₆, for instance, and depositing a passivating layer on the side walls of the etched recess, the etching steps may be connectable periodically, in particular in alternation.

It was discovered according to the exemplary embodiments and/or exemplary methods of the present invention that it is possible to produce tapering microneedles, i.e., microneedles having a negative profile, by reducing the effectiveness of the passivation steps, for instance via a lower gas flow rate and/or shorter passivation periods. In this way the side walls of the structures to be produced are attacked more heavily in the lower region during the patterning steps, and a negative profile is formed.

The method parameters, e.g., the time period for the etching step, the time period for the passivation step, the ratio of etching time to passivation time, the gas flow rate of the etching gas, the gas flow rate of the passivation gas, the ion acceleration, the method duration, may vary widely and must be adapted to each other. The method parameters, in particular, must be adapted as a function of the system utilized for the etching.

Specifically, it was discovered that a negative profile of the microneedles is able to be improved by longer etching periods. The ratio of etching period to passivation period which may lie in a range from 1.5:1 to 5:1, which may be in a range from 2:1 to 3:1.

In an advantageous development according to the exemplary embodiments and/or exemplary methods of the present invention, the time span for the etching step ranges from ≧5 s to ≦30 s, which may be from ≧10 s to ≦20 s. In a further advantageous development according to the exemplary embodiments and/or exemplary methods of the present invention, the time span for the passivation step ranges from ≧1 s to ≦20 s, which may be from ≧2 s to ≦10 s.

In one advantageous development, the exemplary embodiments and/or exemplary methods of the present invention provides that the gas flow rate of the etching gas, such as SF₆, be set to fall within a range from ≧50 sccm to ≦1000 sccm, which may be a range from ≧250 sccm to ≦500 sccm. In another advantageous development, the exemplary embodiments and/or exemplary methods of the present invention provides that the gas flow rate of the passivation gas, such as C₄F₈, be set to fall within a range from ≧50 sccm to ≦500 sccm, which may be a range from ≧100 sccm to ≦200 sccm.

Suitable etching reagents may be gases. Utilizable are etching reagents selected from among the group including NF₃ and/or SF₆; especially, for example, SF₆. Other etching reagents may be selected from the group including ClF₃, BrF₃ and/or XeF₂. Etching reagents selected from among the group including ClF₃, BrF₃ and/or XeF₂ may advantageously cause the etching process to operate isotropically. One advantage that results especially from the use of gaseous etching reagents is the speed of the etching process, the HF-free process control, and the high selectivity, for instance with regard to oxides as masking material.

Passivation means may be selected from among the group including C₄F₈ and/or C₃F₆.

In implementing the method according to the present invention it was discovered that in a further advantageous development the effectiveness of the passivation steps is able to be reduced by greater acceleration of the ions, for instance by increasing the output to triple or quadruple the output in comparison with a production of vertical side walls.

The duration of the microneedle production may range from 15 minutes to 60 minutes, which may be from 30 minutes to 40 minutes.

In an advantageous manner it is possible to provide high etching rates with excellent mask selectivity.

In additional specific embodiments of the method, isotropic etching steps may be provided for the production of tapering microneedles. In this way the structure to be formed may be slimmed even further and the profile of the microneedle optimized further.

Starting from the outer surface, the microneedles taper in the direction of the base of the Si semiconductor substrate.

Starting from the outer surface of the Si semiconductor substrate, the microneedles have a region that has a wider cross-section, while the region of the microneedles that has a smaller cross-section extends in the direction of the base of the Si semiconductor substrate. In contrast to microneedles usually obtained by etching methods and produced by isotropic etching starting from the surface of a Si semiconductor, the microneedles have a “negative” profile.

This offers the advantage that a polymer support layer is able to be applied on the surfaces of the microneedles facing away from the Si semiconductor substrate in a subsequent method step, while the microneedles are still attached to the Si semiconductor substrate by their future tip.

Before the microneedles are affixed on and/or inside a support, the masking layer may optionally be removed. In other exemplary embodiments, the microneedles connected via the webs of the masking layer are able to be attached on and/or inside the support.

Certain exemplary embodiments may use a polymer material as polymer support, which is produced from a thermoplastic polymer selected from among the group including polycarbonate (PC), liquid crystal polymer (LCP), polypropylene styrol, cyclo olefin copolymer (COC) and/or cyclo olefin polymer (COP) and/or mixtures thereof. Polymer materials which are used may be selected from among the group including cyclo olefin copolymer (COC) and/or cyclo olefin polymer (COP). The polymer material may also be made from a duroplastic polymer material. Especially when using cyclo olefin copolymer (COC) and/or cyclo olefin polymer (COP), it is advantageous that these polymers exhibit little swelling and/or fluid absorption and are suitable for delivering medications via microneedles, in particular.

The polymer material need not be degradable, for example. This offers the advantage that the device may remain on the skin for some length of time and can then be removed again.

A polymer support layer is deposited on the microneedle surfaces facing away from the Si semiconductor substrate. In exemplary embodiments, the microneedles are at least partially embedded in the support made from a polymer material, which may be by compression-molding the microneedles and the support, which may be with the aid of methods of hot-stamping or curing, in particular UV-curing.

Hot-stamping within the scope of this invention is understood to denote methods in which the heated Si semiconductor substrate is pressed into the polymer support using a defined force, temperature or displacement characteristic. The term curing, and especially UV-curing, within the meaning of this invention denotes methods in which the Si semiconductor substrate is pressed into a malleable mono-/oligomer solution, the solution then being cured appropriately, in particular by radiation using ultraviolet light (UV).

The polymer support plastically deforms in the process, and the microneedles are pressed into the support. Pressure and temperature are adapted to the particular polymer used. The pressure may lie in a range from ≧50 kPa to ≦7 MPa. The temperature may lie in a range from ≧100° C. to ≦200° C.

Hot-stamping advantageously makes it possible to produce a low-stress structure configuration while maintaining high accuracy. In this way the microneedles may advantageously be transferred into the support with great precision regarding their spacing and alignment, so that the microneedles on a flexible support are able to have the same precise alignment as those on a rigid support, e.g., on a Si semiconductor substrate. Furthermore, the excellent processing capabilities of thin support layers are among the advantages of hot-stamping. In this way highly flexible devices having microneedles are able to be produced on a very thin, flexible support having a thickness that ranges from ≧200 μm to ≦400 μm, for instance.

The desired length of the microneedles projecting from the support may be adjusted by introducing means between the Si semiconductor substrate and the support or by applying means on the Si semiconductor substrate prior to applying the polymer support on the microneedles, which may be used as spacers, e.g., as a spacing layer or spacing foil.

In exemplary embodiments of the present invention, a sacrificial resist is deposited on the Si semiconductor substrate prior to applying the polymer support or prior to compression molding of the microneedles and the polymer support, the layer thickness corresponding to the length of the microneedles that will later project from the support. Sacrificial resists which may be used are selected from among the group including what is known as positive photo resists and/or negative photo resists. Photo resists known as AZ® 9200 or AZ® 4500 and obtainable from the Clariant GmbH or by the trade name of SU8 from Shell Chemical are able to be utilized. The use of positive photo resists and negative photo resists advantageously makes it possible to achieve high layer thicknesses in a range of up to ≧100 μm. In exemplary embodiments, multiple layers of positive photo resists and/or negative photo resists may be provided on the Si semiconductor substrate. The sacrificial resist is removed again after the compression molding. The sacrificial resist may advantageously be chemically dissolved with the aid of acetone, for example, or it may be thermally degradable.

In additional specific embodiments, a polymer layer, in particular a polymer foil, whose layer thickness corresponds to the length of the microneedles later projecting from the support, may be introduced between the Si semiconductor substrate and the polymer support as spacer, by placing it, for example, between the Si semiconductor substrate and the support prior to the step of compression molding.

The polymer material and/or the method parameters in the compression molding are selected such that the polymer layer or polymer foil acting as spacer will not combine with the support irreversibly so that it is removable again following the compression-molding. Polymers having a higher glass temperature than the support and a lower adhesion to silicon, for example, may be used.

Usable are permeable layers or foils, which are pierced by the microneedles during affixation, e.g., the compression molding, or layers or foils, which have appropriately sized holes at the locations of the microneedles.

In additional specific embodiments, structures of the Si semiconductor substrate, which may be provided in the form of a frame, for example, may be used as spacers. The structures of the Si semiconductor substrate are able to be produced by corresponding masking during the etching of the microneedles or in an additional etching step.

It is also possible to set the excursion of the press during the compression molding in such a way that the desired length of the microneedles subsequently projects from the support.

In alternative specific embodiments the microneedles are able to be affixed on the support by bonding the microneedles and support to each other, for instance with the aid of cement. Bonding is especially suitable when using duroplastic polymer materials.

In still other alternative specific embodiments, the polymer support layer is applied by inserting the microneedles in a liquid monomer or oligomer solution of a polymer material. The monomer or oligomer solution may be polymerized out after the microneedles have been embedded. The outpolymerization may be achieved by UV radiation, for instance.

The microneedles are detached from the Si semiconductor substrate after the polymer support has been applied. The microneedles may be detached from the Si semiconductor substrate mechanically, using tensile or transverse forces, for instance.

In an exemplary embodiment, the microneedles may be chemically or electromechanically detached from the Si semiconductor substrate, by etching. In this case the etching reagent attacks at the tip of the microneedles and thereby detaches the microneedles from the Si semiconductor substrate. Etching methods for detaching the microneedles may be selected from among the group that includes isotropic, dry-chemical etching, e.g., using etching reagents selected from among the group including ClF₃, BrF₃ and/or XeF₂, wet-chemical etching, e.g., using HNO₃/H₂O₂-mixtures as etching reagents, or electrochemical etching, using electrolytes containing hydrofluoric acid (HF), in particular. Current densities for the electrochemical etching in hydrous hydrofluoric acid solutions may lie in a range from ≧50 mA/cm² to ≦1000 mA/cm².

The microneedles may also be detached from the Si semiconductor substrate electrically or thermally by annealing through the needle tip.

In additional exemplary embodiments, microneedles in the form of a hollow needle having a continuous channel are able to be produced. A hollow needle may be produced in that a channel is formed through the structure of the future microneedle by isotropic etching of the Si semiconductor substrate. Methods may include dry-etching methods, in particular trenching methods, e.g., the trenching method known as plasma reactive ion etching (plasma RIE), or deep-trenching methods; especially suitable is what is commonly known as the Bosch process.

In exemplary embodiments, a continuous channel is likewise charged in advance in the support, thereby making it possible to provide a continuous opening through microneedles and support. The continuous channel through the support may be charged in advance prior to the compression-molding, or it may be introduced after the compression-molding.

Such an access to the microneedles makes it possible to deliver substances or active ingredients into or under the skin through the support and through the microneedles. A drug depot, for instance, is able to be set up above the device.

In exemplary embodiments, at least partially or completely porous microneedles may be produced. In exemplary embodiments of the method according to the present invention it is therefore possible to produce microneedles that are rendered porous. The microneedle may be rendered porous by electrochemical anodizing. Anodic, electrochemical etching processes utilize the Si semiconductor substrate, such as a silicon wafer, as anode.

The porosification may be implemented in electrolytes containing hydrofluoric acid, in particular hydrous hydrofluoric acid solutions, or mixtures containing hydrofluoric acid, water and additional reagents, in particular selected from among the group including wetting agents such as alcohols, may be selected from among the group including ethanol and/or isopropanol, and/or relaxants such as surfactants.

The hydrofluoric acid content of a hydrous hydrofluoric acid solution may lie in a range from ≧5 vol.-% to ≦40 vol.-% in relation to the total volume of the electrolyte. A wetting agent may be added for better method control. Wetting agents may be selected from among the group including isopropanol and/or ethanol. Current densities may range from ≧10 mA/cm² to ≦400 mA/cm², and may be from ≧50 mA/cm² to ≦250 mA/cm².

P-doped Si semiconductor substrates may be used. The microstructure of the microneedle is able to be influenced in an advantageous manner by the selection of the doping. The use of doping of less than 10¹⁷/cm³ may be provided, this indicated number corresponding to the doping atoms per cm³ of the Si semiconductor substrate. This makes it possible to obtain a nanoporous structure. The pore diameter in a nanoporous structure may range from ≧0.5 nm to ≦5 nm. Also, the use of doping of more than 10¹⁷/cm³ may be provided, which allows a mesoporous structure to be obtained whose pore diameter may lie in a range from ≧10 nm to ≦20 nm. The advantage of a nanoporous or mesoporous structure of the porosity of the microneedle is that substances or active ingredients to be delivered into or through the skin, for example, are able to be delivered under the skin without an inner channel in the microneedle, by impregnating the microneedle with the substance or the active ingredient.

The pororization may be implemented at different times during the production process. For example, the Si semiconductor substrate may be rendered porous first before applying and patterning a masking layer. This enables the production of porosified microneedles by the etching process in step c). Porosification, which may be done before patterning, makes it possible to produce completely porosified microneedles.

In additional developments, the Si semiconductor substrate and the microneedles may be rendered porous once the microneedles have been patterned. In this way, completely or partially porosified microneedles may be produced, such as microneedles having a layer that is rendered porous.

In other developments, the Si semiconductor substrate and the microneedles may be porosified after the microneedles have been pressed into the support. This is possible, for instance, by providing an access to the microneedles through the support, the support may be resistant to the hydrofluoric acid which may be used. This embodiment makes it possible to detach the microneedles from the Si semiconductor substrate after porosification employing what is known as electropolishing. Electropolishing means that the porosity is increased by varying the etching parameters, in particular by increasing the current density or reducing the hydrofluoric acid concentration to such an extent that it reaches 100% in the region of the needle tip. A porosity of 100% corresponds to the complete dissolution of the Si semiconductor material in this region. In this embodiment the Si semiconductor substrate need not be doped in the region that corresponds to the future tip of the microneedles, whereas doping is carried out in the region that corresponds to the future body of the microneedles.

It is especially advantageous in this context that the Si semiconductor substrate is not destroyed but is available again for another implementation of the present method.

In still other developments, the microneedles may be rendered porous after having been detached from the Si semiconductor substrate.

The indicated sequence of the method steps b) through h) is not to be understood in the sense of a fixed sequence within the scope of the exemplary embodiments and/or exemplary methods of the present invention. Depending on the selected timing of the porosification of the Si semiconductor substrate and/or the microneedles, the sequence of the method steps b) through h) may vary accordingly.

In exemplary embodiments the microneedles may be developed to be at least partially porous, which may be completely porous. The microneedles may have a porous design in specific regions such as the tip of the microneedles, or the microneedles may have a porous layer. It is also possible that the entire microneedles have a porous design.

On the one hand, a porous structure of the microneedles may offer the advantage that the microneedles become permeable to the substances to be applied or that they are able to store the substances. Another advantage of microneedles made of silicon and rendered porous is the increased biocompatibility of the microneedles. Fragments of the microneedles may be broken down in the body, harmless silicic acid being produced in the process.

The thickness of a porous layer of the microneedles may vary widely depending on the requirements; for example, it is possible to porosify only a thin surface layer, or the porous layer may have a thickness of several 10 μm. The thickness of the porous layer may range from ≧0.5 μm to ≦50 μm, which may be from ≧1 μm to ≦20 μm, and which may be from ≧3 μm to ≦15 μm.

The porosity of the microneedles may lie in a range from ≧10% to ≦80%, and may be in a range from ≧25% to ≦55%. A porosity of the microneedles of less than 55% may advantageously provide sufficient mechanical stability of the microneedles.

In the exemplary embodiments and/or exemplary methods of the present invention, “porosity” is defined in such a manner that it indicates the empty space within the pattern and the remaining substrate material. It may either be determined optically, i.e., from an evaluation of, e.g., microscopic photographs, or chemically. In the case of chemical determination, the following applies:

Porosity P=(m1−m2)/(m1−m3), m1 being the mass of the sample prior to porosification, m2 being the mass of the sample after porosification, and m3 being the mass of the sample after etching it with a 1 molar NaOH solution that chemically dissolves the porous structure. As an alternative, the porous structure may also be dissolved by a KOH/isopropanol solution.

Furthermore, the microneedles may have different pore structures; the pore size may range from a few nanometers to ≧50 nm in diameter. For instance, pores having a diameter of ≦5 nm, in the range from between ≧5 nm to ≦50 nm, or ≧50 nm may be used.

Porous microneedles may offer the advantage that the substances to be dispensed, especially pharmaceutical substances, may be stored in the porous needle material. These substances are then able to be delivered through the skin in a time-delayed manner, for example. This embodiment is particularly suitable for highly efficacious active ingredients to be dispensed in low doses, such as vaccines.

In exemplary embodiments, the microneedles made of silicon have at least one continuous channel. The microneedles may have one or several channels. A channel is a hollow passage that extends axially through the microneedle, so that a “hollow needle” is formed. The term “hollow needle” within the scope of this invention means that the microneedle has a continuous opening or a continuous channel through the interior of the microneedle structure. This may offer the advantage that the substances to be delivered, especially pharmaceutical agents, are able to be delivered through the microneedles, without the substance having to be stored in the microneedle. For instance, a gel containing the substances, an ointment, a plaster containing active ingredients, or a similar device may be applied on the device.

Porosified hollow needles and/or porosified microneedles without a channel running through the interior of the microneedle structure are able to be used.

The exemplary embodiments and/or exemplary methods of the present invention also relates to a device which is suitable for delivering a substance into or through the skin and which includes an array of microneedles developed out of an Si semiconductor substrate, the microneedles being at least partially embedded in a flexible support made from a polymer material via the region of the microneedles having a wider cross-section, or being affixed on the flexible carrier via the region of the microneedles having a wider cross-section, the device being producible according to the method of the present invention.

The device, which includes an array of microneedles situated on a flexible support made from a polymer material is capable of adapting to the form of the patient's skin area on which it is applied, and of providing a more uniform penetration depth of the microneedles. Furthermore, due to the flexible structure of the support, the device is able to adapt to the patient's movements and the related shifting of the skin. The device may remain on the patient's skin without being perceived as an inflexible and irritating foreign body.

The device according to the present invention having an array of microneedles on a support may have a size ranging from ≧1 mm² to ≦30 mm², which may range from ≧2 mm² to ≦5 mm². A device according to the present invention may have microneedles whose number ranges from ≧1 to ≦4000 microneedles, and may be from ≧25 to ≦400 microneedles.

The thickness of the support may vary widely and is adaptable to the device's intended use. The support may have a thickness ranging from ≧200 μm to ≦1 mm, may range from ≧400 μm to ≦600 μm. For example, especially for uses where the array is to be pressed onto the skin only briefly and then removed again, a polymer support having a thickness in a range from ≧500 μm to ≦1 mm may be used, which is able to offer reliable handling despite the rapid movement that is required in this context. Depending on the material, thicker supports may become inflexible and possibly exhibit more pronounced swelling due to an absorption of liquid.

However, especially for uses where the device is to remain on the skin, the support may have a thickness ranging from ≧200 μm to ≦400 μm, which may range from ≧250 μm to ≦350 μm. With thinner supports there is a risk of tearing during use. The support may be a polymer foil, for example.

The total length of the microneedles may range from ≧150 μm to ≦500 μm, or from ≧200 μm to ≦400 μm, or from ≧250 μm to ≦350 μm.

The depth at which the microneedles project into the support may range from ≧50 μm to ≦150 μm, or from ≧100 μm to ≦120 μm. If a smaller portion of the microneedles is anchored or embedded in the support, there is the risk that the microneedles become detached or break off when the device is pressed into the skin or removed therefrom.

This depth at which the microneedles project into the support may provide secure anchoring of the microneedles in the support. More specifically, a depth ranging from ≧50 μm to ≦150 μm may ensure that the microneedles together with the support also are removed from the skin again when the device is taken off, so that all or virtually all of the microneedles are removed from the skin.

The length of the microneedles projecting from the support may range from ≧80 μm to ≦300 μm, and may be from ≧120 μm to ≦250 μm.

The average diameter of the needle tip may range from ≧1 μm to ≦50 μm, or from ≧2 μm to ≦30 μm, or from ≧4 μm to ≦20 μm.

The microneedles may be disposed on and/or inside the support at regular intervals. A uniform setup may offer the particular advantage that the active ingredient to be applied is given uniform access.

Usable microneedles may have various cross-sectional forms, such as round, angular or star-shaped. Furthermore, the microneedles may be placed on the support in a symmetrical or asymmetrical arrangement. The outside of the microneedles, especially if hollow needles are involved, may have a concave design. This makes it possible to apply a substance to be delivered along the outer surface of the needle.

For example, the device according to the present invention is suitable for the application of active ingredients selected from among the group that includes dermatics, cardiatics, hormones including insulin and contraceptives, anticoagulants, anti-emetics, analgesics, anti-depressants, anti-arrhythmic drugs, anti-psychotics, anxiolytic agents, anti-Parkinson drugs, anti-osteoporosic drugs, antibiotics, serums, anti-allergic agents, and/or vaccines. In addition, the device according to the present invention is suitable, for instance, for the application of substances selected from among the group including nucleic acids, in particular DNA and/or peptides.

In exemplary embodiments, the support has continuous accesses or channels, which form an access to the microneedles. In exemplary embodiments, the support has continuous accesses or channels, which give the substances to be delivered access to porosified microneedles that let a supplied substance pass through the pores, or to continuous channels of the microneedles.

In addition, the use of a device as a delivery unit for substances into or through the skin is subject matter of the exemplary embodiments and/or exemplary methods of the present invention. It advantageously permits the delivery of substances or active ingredients without requiring injections.

What is claimed is:

1. A method for producing a device having an array of microneedles, the microneedles being affixed to a flexible support made from a polymer material, the method comprising:
a) supplying a Si semiconductor substrate;
b) applying and patterning a masking layer on a surface of the Si semiconductor substrate, openings in the masking layer being provided through which an etching reagent acts on the Si semiconductor substrate;
c) developing the array of microneedles out of the Si semiconductor substrate, which taper from an outer surface in a direction of a base of the Si semiconductor substrate, with the aid of micromechanical patterning through the masking layer using wet or dry chemical etching;
(f) depositing a cohesive polymer support layer on the microneedle surfaces facing away from the Si semiconductor substrate; and
(g) detaching the microneedles from the Si semiconductor substrate.

2. The method of claim 1, wherein the openings in the masking layer are separated by webs formed in the masking layer in the form of a grid.

3. The method of claim, wherein tapering microneedles are developed out of the Si semiconductor substrate by deep-etching using alternating etching and passivation steps by a plasma, a ratio of etching time to passivation time ranging from 1.5:1 to 5:1.

4. The method of claim 1, wherein the microneedles are partially embedded in a support by compression-molding the microneedles and the polymer support, using hot-stamping or UV curing.

5. The method of claim 1, wherein a length of the microneedles projecting from the support is adjusted by applying a sacrificial resist on the Si semiconductor substrate prior to the compression molding of the microneedles and the polymer support, whose layer thickness corresponds to the length of the microneedles later projecting from the support, and removing the resist again following the pressing in.

6. The method of claim 1, wherein a polymer foil layer, whose layer thickness corresponds to the length of the microneedles later projecting from the support, is inserted between the Si semiconductor substrate and the polymer support, and then is removed again after the pressing in.

7. The method of claim 1, wherein a polymer material formed from a at least one thermoplastic polymer selected from among polycarbonate, liquid crystal polymer, polypropylene styrol, cyclo olefin copolymer, cyclo olefin polymer, and mixtures thereof, and a duroplastic polymer material is used as a polymer support.

8. The method of claim 3, wherein a time span for the etching step is: (i) set to range from ≧5 s to ≦30 s, and (ii) set to range from ≧10 s to ≦20 s; and wherein the time span for the passivation step is: (i) set to range from ≧1 s to ≦20 s, and set to range from ≧2 s to ≦10 s.

9. A device for delivering a substance into or through the skin, comprising:
a flexible support; and
an array of microneedles developed out of a Si semiconductor substrate, the microneedles being (i) at least partially embedded in the flexible support made from a polymer material via a region of the microneedles having a wider cross-section, or (ii) affixed on the flexible support via the region of the microneedles having the wider cross-section.

10. The device of claim 9, wherein the support has a thickness that: (i) ranges from ≧200 μm to ≦1 mm; or (ii) ranges from ≧400 μm to ≦600 μm.

11. The device of claim 9, wherein the length at which the microneedles project from the support (i) ranges from ≧80 μm to ≦300 μm, or (ii) ranges from ≧120 μm to ≦250 μm.

12. The method of claim 1, further comprising:
d) removing the masking layer; and
e) rendering porous at least one of the Si semiconductor substrate and the microneedles.
